(19) United States
(12) Patent Application Publication
(10) Pub. No.: US 2024/0177834 A1
(43) Pub. Date: May 30, 2024

(54) IMAGE PROCESSING DEVICE, IMAGE PROCESSING SYSTEM, IMAGE PROCESSING METHOD, AND IMAGE PROCESSING PROGRAM

(71) Applicant: TERUMO KABUSHIKI KAISHA, Tokyo (JP)

(72) Inventors: Shunsuke YOSHIZAWA, Ebina-shi (JP); Yasukazu SAKAMOTO, Hiratsuka-shi (JP); Katsuhiko SHIMIZU, Fujinomiya-shi (JP); Hiroyuki ISHIHARA, Tokyo (JP)

(73) Assignee: TERUMO KABUSHIKI KAISHA, Tokyo (JP)

(21) Appl. No.: 18/428,352

(22) Filed: Jan. 31, 2024

Related U.S. Application Data
(63) Continuation of application No. PCT/JP2022/029541, filed on Aug. 1, 2022.

(30) Foreign Application Priority Data
Aug. 2, 2021 (JP) 2021-126943

(51) Int. Cl. G16H 30/40; A61B 8/08; A61B 34/20; G06T 7/00

(52) U.S. Cl. CPC G16H 30/40; A61B 8/483; A61B 34/20; G06T 7/0012; A61B 2034/2055; G06T 2207/30048

(57) ABSTRACT

An image processing device includes a control unit configured to determine, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image, and specify a position of the medical device based on a position of the deformation spot when it is determined that the deformation spot is included in the at least one cross-sectional image.

### CROSS-REFERENCES TO RELATED APPLICATIONS

This application is a continuation of International Application No. PCT/JP2022/029541 filed on Aug. 1, 2022, which claims priority to Japanese Application No. 2021-126943 filed on Aug. 2, 2021, the entire content of both of which is incorporated herein by reference.

### TECHNOLOGICAL FIELD

The present disclosure relates to an image processing device, an image processing system, an image processing method, and an image processing program.

### BACKGROUND DISCUSSION

U.S. Patent Application Publication No. 2010/0215238 and U.S. Pat. Nos. 6,385,332 and 6,251,072 disclose a technique of generating a three-dimensional image of a cardiac cavity or a blood vessel by using an ultrasound image system.

Treatment using intravascular ultrasound (IVUS) is widely executed for a cardiac cavity, a cardiac blood vessel, a lower limb artery region, and the like. IVUS is a device or a method for providing a two-dimensional image of a plane perpendicular to a longitudinal axis of a catheter.

At present, an operator needs to execute treatment while reconstructing a three-dimensional structure by stacking the two-dimensional images of IVUS in his/her head, which can be a barrier particularly to young doctors or inexperienced doctors. In order to remove such a barrier, it is conceivable to automatically generate a three-dimensional image expressing a structure of a biological tissue such as the cardiac cavity or the blood vessel from the two-dimensional images of IVUS and display the generated three-dimensional image toward the operator. Since the operator can see only an outer wall of the tissue only by displaying the generated three-dimensional image as it is, it is conceivable to cut out a part of the structure of the biological tissue in the three-dimensional image so that a lumen can be viewed. When a catheter different from an IVUS catheter, such as an ablation catheter or a catheter for atrial septal puncture, is inserted into the biological tissue, it is conceivable to further display a three-dimensional image expressing the different catheter.

However, when the operator takes an action, such as pressing the catheter against a fossa ovalis, the catheter can be stuck in the tissue, and it can be difficult to detect a distal end of the catheter from the two-dimensional image of IVUS. As a result, a three-dimensional image in which the distal end of the catheter is cut out is drawn. Therefore, the operator cannot see the distal end of the catheter, and the treatment using the catheter cannot be smoothly executed.

### SUMMARY

The present disclosure can enable specification of a position of an elongated medical device even when it is difficult to detect the medical device from an image obtained using a sensor.

An image processing device in an aspect of the present disclosure includes a control unit configured to determine, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image, and specify a position of the medical device based on a position of the deformation spot when it is determined that the deformation spot is included in the at least one cross-sectional image.

In an embodiment, the control unit is configured to determine, when the input of the at least one cross-sectional image is received, whether the medical device is included in the at least one cross-sectional image, and determine, when it is determined that the medical device is not included in the at least one cross-sectional image, whether the deformation spot is included in the at least one cross-sectional image.

In an embodiment, when it is determined that the medical device is not included in the at least one cross-sectional image and it is determined that the deformation spot is included in the at least one cross-sectional image, the control unit is configured to specify a position of the medical device in a three-dimensional space based on a position of the deformation spot in the at least one cross-sectional image, and when it is determined that the medical device is included in the at least one cross-sectional image, the control unit is configured to specify the position of the medical device in the three-dimensional space based on the position of the medical device in the at least one cross-sectional image.

In an embodiment, the control unit is configured to cause a display to display a three-dimensional object group including an object representing the biological tissue in the three-dimensional space and an object representing the medical device in the three-dimensional space.

In an embodiment, the control unit is configured to determine whether the deformation spot is included in the at least one cross-sectional image by selecting at least one pixel from a pixel group of the at least one cross-sectional image and determining whether the selected pixel corresponds to the deformation spot.

In an embodiment, the control unit is configured to calculate, for each pixel of the at least one cross-sectional image, similarity with a target pixel for each pixel present on a circumference at a constant distance from the target pixel, and select the target pixel as the at least one pixel when an angle at which pixels having the calculated similarity equal to or greater than a reference value continuously present on the circumference is within a reference range.

In an embodiment, the control unit is configured to extract, for each pixel on a boundary line corresponding to the inner surface of the biological tissue in the pixel group of the at least one cross-sectional image, a first pixel group present on one side of a target pixel on a boundary line and a second pixel group present on the other side of the target pixel on the boundary line, and select the target pixel as the at least one pixel when magnitude of an angle formed by a regression line corresponding to the extracted first pixel group and a regression line corresponding to the extracted second pixel group is within a reference range.

In an embodiment, the control unit is configured to input the at least one cross-sectional image to a trained model that infers, using an image obtained using the sensor as an input, a pixel corresponding to the deformation spot of the input image, and select the inferred pixel as the at least one pixel.

In an embodiment, the control unit is configured to determine whether the selected pixel corresponds to the deformation spot according to a positional relationship between the selected pixel and the medical device in a cross-sectional image including the medical device different from the at least one cross-sectional image.

In an embodiment, the control unit is configured to select the at least one pixel from a pixel group of each of two or more cross-sectional images continuously obtained using the sensor, and determine whether the selected pixel corresponds to the deformation spot according to a difference in position of the selected pixel between the two or more cross-sectional images.

In an embodiment, the control unit is configured to input the at least one cross-sectional image to a trained model that infers, using an image obtained using the sensor as an input, whether the deformation spot is included in the input image, and determine whether the deformation spot is included in the at least one cross-sectional image by referring to an obtained inference result.

In an embodiment, the control unit is configured to input the at least one cross-sectional image to a trained model configured to infer, using an image obtained using the sensor as an input, data indicating a position of the deformation spot in the input image, and determine whether the deformation spot is included in the at least one cross-sectional image by referring to an obtained inference result.

An image processing system in an aspect of the present disclosure includes the image processing device and the sensor.

An image processing system in an aspect of the present disclosure includes the image processing device and the display.

An image processing method in an aspect of the present disclosure includes: determining, by a control unit, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image; and specifying, by the control unit, a position of the medical device based on a position of the deformation spot when it is determined that the deformation spot is included in the at least one cross-sectional image.

A non-transitory computer-readable medium storing an image processing program in an aspect of the present disclosure causes a computer to execute a process comprising: determining, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image; and specifying a position of the medical device based on a position of the deformation spot when it is determined that the deformation spot is included in the at least one cross-sectional image.

According to the present disclosure, it is possible to specify a position of an elongated medical device even when it is difficult to detect the medical device from an image obtained using a sensor.

### BRIEF DESCRIPTION OF THE DRAWINGS

FIG. 1 is a perspective view of an image processing system according to an embodiment of the present disclosure.

FIG. 2 is a diagram illustrating an example of a three-dimensional image and a cross-sectional image displayed on a display by the image processing system according to the embodiment of the present disclosure.

FIG. 3 is a diagram illustrating an example of a cutting region formed by the image processing system according to the embodiment of the present disclosure.

FIG. 4 is a block diagram illustrating a configuration of an image processing device according to the embodiment of the present disclosure.

FIG. 5 is a perspective view of a probe and a drive unit according to the embodiment of the present disclosure.

FIG. 6 is a flowchart illustrating an operation of the image processing system according to the embodiment of the present disclosure.

FIG. 7 is a flowchart illustrating an operation of the image processing system according to the embodiment of the present disclosure.

FIG. 8 is a diagram illustrating an example of a selection method used by the image processing system according to the embodiment of the present disclosure.

FIG. 9 is a diagram illustrating an example of a selection method used by the image processing system according to the embodiment of the present disclosure.

FIG. 10 is a diagram illustrating an example of a determination method used by the image processing system according to the embodiment of the present disclosure.

FIG. 11 is a diagram illustrating an example of a determination method used by the image processing system according to the embodiment of the present disclosure.

### DETAILED DESCRIPTION

Set forth below with reference to the accompanying drawings is a detailed description of embodiments of an image processing device, an image processing system, an image processing method, and an image processing program.

In the drawings, the same or corresponding parts are denoted by the same reference numerals. In the description of each embodiment, the description of the same or corresponding parts will be omitted or simplified as appropriate.

An embodiment of the present disclosure will be described.

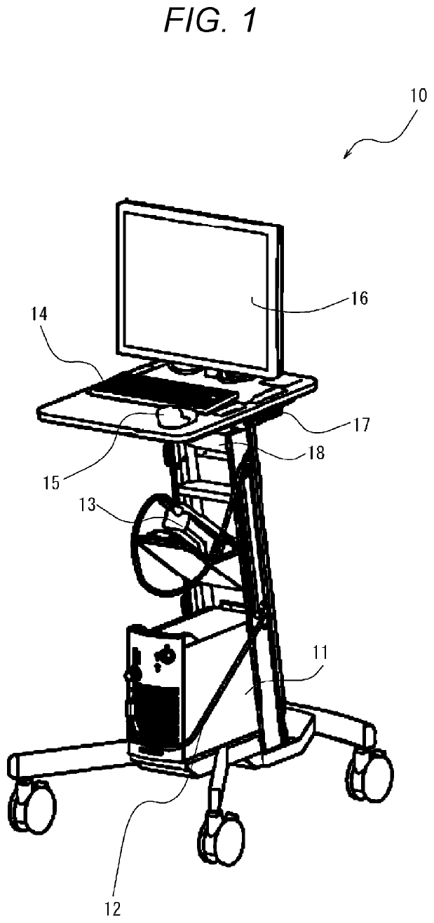

An outline of the present embodiment will be described with reference to FIGS. 1 to 4.

An image processing device 11 according to the present embodiment is a computer that causes a display 16 to display at least one cross-sectional image included in tomographic data 51 that is a data set obtained using a sensor moving in a lumen 61 of a biological tissue 60. That is, the image processing device 11 causes the display 16 to display at least one cross-sectional image obtained using the sensor. Specifically, the image processing device 11 causes the display 16 to display a cross-sectional image 54 representing a cross section 64 of the biological tissue 60 orthogonal to a movement direction of the sensor.

When receiving an input of the cross-sectional image 54, the image processing device 11 determines whether a deformation spot 69 that is on an inner surface 65 of the biological tissue 60 and that has been deformed by pressing a catheter 63 inserted into the lumen 61 is included in the cross-sectional image 54. When it is determined that the deformation spot 69 is included in the cross-sectional image 54, the image processing device 11 specifies a position of the catheter 63 based on a position of the deformation spot 69.

In the present embodiment, when receiving the input of the cross-sectional image 54, the image processing device 11 determines whether the catheter 63 is included in the cross-sectional image 54. When it is determined that the catheter 63 is not included in the cross-sectional image 54, the image processing device 11 determines whether the deformation spot 69 is included in the cross-sectional image 54. In a modification of the present embodiment, the image processing device 11 may determine whether the deformation spot 69 is included in the cross-sectional image 54 regardless of whether the catheter 63 is included in the cross-sectional image 54.

When it is determined that the catheter 63 is not included in the cross-sectional image 54 and it is determined that the deformation spot 69 is included in the cross-sectional image 54, the image processing device 11 specifies a position of the catheter 63 in a three-dimensional space based on the position of the deformation spot 69 in the cross-sectional image 54. Then, the image processing device 11 causes the display 16 to display a three-dimensional object group including an object representing the biological tissue 60 in the three-dimensional space and an object representing the catheter 63 in the three-dimensional space.

In the present embodiment, when it is determined that the catheter 63 is included in the cross-sectional image 54, the image processing device 11 specifies the position of the catheter 63 in the three-dimensional space based on the position of the catheter 63 in the cross-sectional image 54. Then, the image processing device 11 causes the display 16 to display the three-dimensional object group including the object representing the biological tissue 60 in the three-dimensional space and the object representing the catheter 63 in the three-dimensional space.

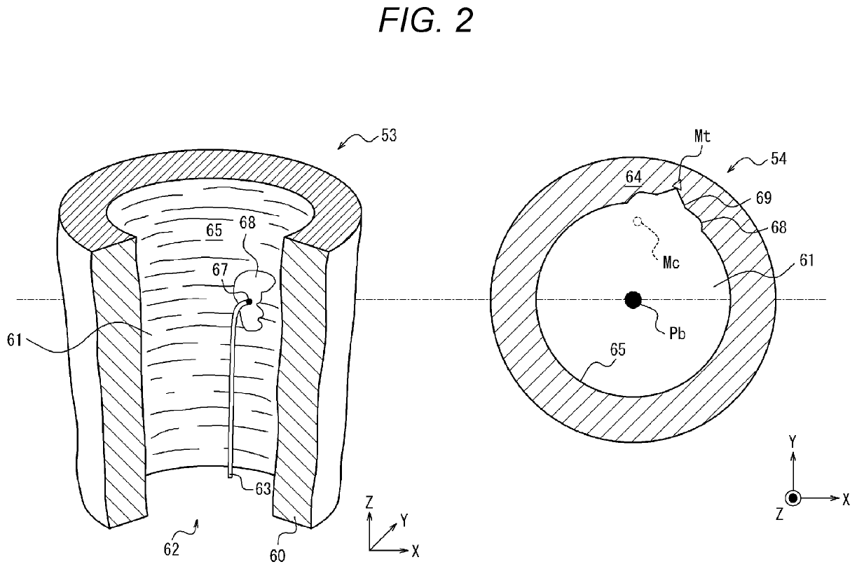

In the example of FIG. 2, the deformation spot 69 can be a tent-like extending part when the catheter 63 is pressed against a fossa ovalis 68 and the fossa ovalis 68 extends like a tent during treatment of atrial septal puncture, that is, a tenting spot having conical or triangular shape. The image processing device 11 causes the display 16 to display a mark Mt indicating a position of the tenting spot in the cross-sectional image 54. The mark Mt may be a mark of any color and shape, but is, for example, a solid yellow triangle in the example of FIG. 2. The mark Mt is directed in an ultrasound wave emission direction from a centroid Pb of the cross section 64 of the biological tissue 60 into which the catheter 63 that is an elongated medical device is inserted. Therefore, a user can rather easily recognize that the mark Mt is a distal end object representing a distal end 67 of the catheter 63 causing tenting. Since a tissue is conically recessed at the time of tenting, the tenting spot is basically displayed continuously for several frames. It is possible to specify a position of the distal end 67 of the catheter 63 stuck in the tissue by determining the tenting spot based on information indicating a position of the catheter 63 detected last in time series or in a Z-axis direction by the image processing device 11. Once the position of the tenting spot is specified, the position of the distal end 67 of the catheter 63 stuck in the tissue can be specified by determining the tenting spot based on information indicating a position of the tenting spot specified in the latest frame or a previous frame instead of the information indicating the position of the catheter 63. In a modification of the present embodiment, a catheter object connected to the tenting spot may be displayed on the cross-sectional image 54. In such a modification, a color tone of the catheter object may be changed according to a distance in the Z-axis direction from the cross-sectional image 54, and a direction of the triangle of the mark Mt may be made to coincide with a direction from a connection point of the catheter object connected to the tenting spot toward the tenting spot. In that case, a user can more easily recognize that the mark Mt is the distal end object representing the distal end 67 of the catheter 63 causing tenting.

The center of the tenting spot in the three-dimensional space may be visualized two-dimensionally by softening the color according to a distance in the Z-axis direction from the center of the tenting spot, that is, an apex of the conically recessed tissue. The center of the tenting spot can be, for example, at a position farthest in a radial direction from the center of the cross-sectional image 54 or the centroid Pb of the cross section 64 in the tenting spot.

In the example of FIG. 2, the image processing device 11 further causes the display 16 to display a mark Mc indicating a position of the catheter 63 specified in a frame in which the catheter 63 has been detected last, such as the previous frame. The mark Mc may be a mark of any color and shape that can be distinguished from the mark Mt, but is, for example, a dotted red circle in the example of FIG. 2.

In the example of FIG. 2, the image processing device 11 causes the display 16 to display the distal end 67 of the catheter 63 stuck in (or pushed into) the tissue at the time of tenting in a color different from that of a catheter tube created based on the information indicating the detected position of the catheter 63 even in three dimensions.

According to the present embodiment, it is possible to specify the position of the elongated medical device such as the catheter 63 even when it is difficult to detect the medical device from the cross-sectional image 54 obtained using the sensor. For example, when an operator takes an action, such as pressing the catheter 63 against the fossa ovalis 68, the catheter 63 gets stuck in (or pushed into) the tissue, and it can be difficult to detect the distal end 67 of the catheter 63 from the cross-sectional image 54. However, in the present embodiment, when a tenting spot can be detected from the cross-sectional image 54, the distal end 67 of the catheter 63 can be detected based on a position of the tenting spot. As a result, a three-dimensional image 53 including the distal end 67 of the catheter 63 can be drawn. Therefore, the operator can see the distal end 67 of the catheter 63, and treatment using the catheter 63 can be rather smoothly executed. In a modification of the present embodiment, an elongated medical device other than the catheter 63, such as a guide wire or a transseptal needle, may be detected by a method similar to that of the present embodiment.

In the present embodiment, the image processing device 11 generates and updates three-dimensional data 52 representing the biological tissue 60 with reference to the tomographic data 51 that is the data set obtained using the sensor. The image processing device 11 causes the display 16 to display the three-dimensional data 52 as the three-dimensional image 53 together with the cross-sectional image 54. That is, the image processing device 11 causes the display 16 to display the three-dimensional image 53 and the cross-sectional image 54 with reference to the tomographic data 51.

The image processing device 11 forms, in the three-dimensional data 52, an opening 62 for exposing the lumen 61 of the biological tissue 60 in the three-dimensional image 53. In the example of FIG. 2, the opening 62 is formed so that the catheter 63 including the distal end 67 and the fossa ovalis 68 can be seen. Then, a viewpoint when the three-dimensional image 53 is displayed on a screen is adjusted according to a position of the opening 62. The viewpoint is a position of a virtual camera disposed in the three-dimensional space.

According to the present embodiment, a part of a structure of the biological tissue 60 is cut out in the three-dimensional image 53, so that the lumen 61 of the biological tissue 60 can be viewed.

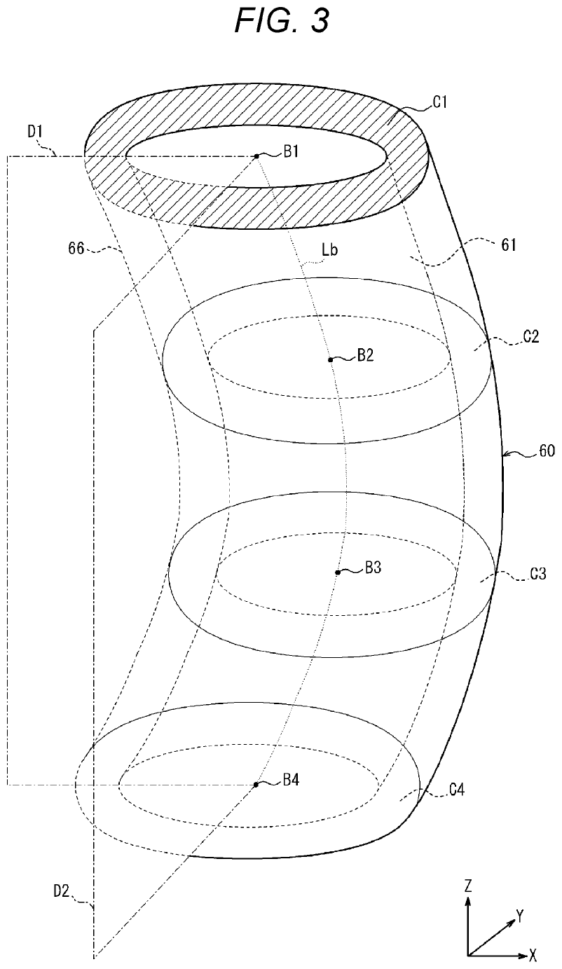

The biological tissue 60 can include, for example, an organ such as a blood vessel or a heart. The biological tissue 60 is not limited to only an anatomically single organ or a part the anatomically single organ, but also includes a tissue having a lumen across a plurality of organs. An example of such a tissue can be, specifically, a part of a vascular tissue extending from an upper part of an inferior vena cava to a lower part of a superior vena cava through a right atrium. In the example of FIG. 3, the biological tissue 60 is a blood vessel.

In FIG. 2, the Z direction corresponds to the movement direction of the sensor, but as illustrated in FIG. 3, for convenience, the Z direction may be regarded as corresponding to a longitudinal direction of the lumen 61 of the biological tissue 60. An X direction orthogonal to the Z direction and a Y direction orthogonal to the Z direction and the X direction may be regarded as corresponding to lateral directions of the lumen 61 of the biological tissue 60.

In the example of FIG. 3, the image processing device 11 calculates positions of centroids B1, B2, B3, and B4 of cross sections C1, C2, C3, and C4 of the biological tissue 60 by using the three-dimensional data 52. The image processing device 11 sets a pair of planes intersecting at a single line Lb passing through the positions of the centroids B1, B2, B3, and B4 as cutting planes D1 and D2. The image processing device 11 forms, in the three-dimensional data 52, a region interposed between the cutting planes D1 and D2 in the three-dimensional image 53 and exposing the lumen 61 of the biological tissue 60, as a cutting region 66. In the three-dimensional image 53, the opening 62 as illustrated in FIG. 2 is formed by the cutting region 66 being set to be hidden or transparent.

In the case of a three-dimensional model of the bent blood vessel as illustrated in FIG. 3, when the three-dimensional model is cut with one plane to display the lumen 61, there is a case in which an inside of the blood vessel cannot be correctly displayed. In the present embodiment, as illustrated in FIG. 3, by continuously capturing centroids of the blood vessel, the three-dimensional model can be cut such that the inside of the blood vessel can be reliably displayed.

In FIG. 3, for convenience, the four cross sections C1, C2, C3, and C4 are illustrated as the plurality of cross sections of the biological tissue 60 orthogonal to the Z direction, but the number of cross sections serving as calculation targets of the centroid positions is not limited to four, and is preferably the same as the number of cross-sectional images acquired by IVUS.

A configuration of an image processing system 10 according to the present embodiment will be described with reference to FIG. 1.

The image processing system 10 can include the image processing device 11, a cable 12, a drive unit 13, a keyboard 14, a mouse 15, and the display 16.

The image processing device 11 can be a dedicated computer specialized for image diagnosis in the present embodiment, but may also be a general-purpose computer such as a personal computer (PC).

The cable 12 is used to connect the image processing device 11 and the drive unit 13.

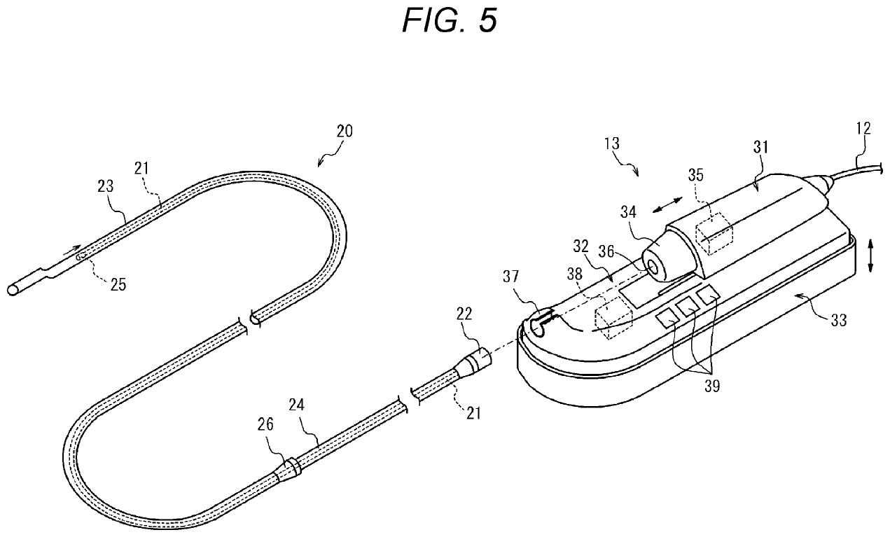

The drive unit 13 is a device to be used by connecting to a probe 20 illustrated in FIG. 5 to drive the probe 20. The drive unit 13 can also be referred to as motor drive unit (MDU). The probe 20 is applied to IVUS. The probe 20 can also be referred to as an IVUS catheter or an image diagnostic catheter.

The keyboard 14, the mouse 15, and the display 16 are connected to the image processing device 11 via any cable or wirelessly. The display 16 can be, for example, a liquid crystal display (LCD), an organic electro luminescence (EL) display, or a head-mounted display (HMD).

The image processing system 10 optionally further includes a connection terminal 17 and a cart unit 18.

The connection terminal 17 is used to connect the image processing device 11 and an external device. The connection terminal 17 can be, for example, a universal serial bus (USB) terminal. The external device can be, for example, a recording medium such as a magnetic disc drive, a magneto-optical disc drive, or an optical disc drive.

The cart unit 18 can be, for example, a cart equipped with casters for movement. The image processing device 11, the cable 12, and the drive unit 13 are disposed on a cart body of the cart unit 18. The keyboard 14, the mouse 15, and the display 16 are disposed on an uppermost table of the cart unit 18.

Configurations of the probe 20 and the drive unit 13 according to the present embodiment will be described with reference to FIG. 5.

The probe 20 can include a drive shaft 21, a hub 22, a sheath 23, an outer tube 24, an ultrasound transducer 25, and a relay connector 26.

The drive shaft 21 passes through the sheath 23 to be inserted into a body cavity of a living body and the outer tube 24 connected to a proximal end of the sheath 23, and extends to an inside of the hub 22 disposed at a proximal end of the probe 20. The drive shaft 21 is provided with the ultrasound transducer 25, which transmits and receives signals, at a distal end of the drive shaft 21, and is rotatably disposed in the sheath 23 and the outer tube 24. The relay connector 26 connects the sheath 23 and the outer tube 24.

The hub 22, the drive shaft 21, and the ultrasound transducer 25 are connected to each other to integrally move forward and backward in an axial direction. Therefore, for example, when the hub 22 is pressed toward a distal side, the drive shaft 21 and the ultrasound transducer 25 move inside the sheath 23 toward the distal side. For example, when the hub 22 is pulled toward a proximal side, the drive shaft 21 and the ultrasound transducer 25 move inside the sheath 23 toward the proximal side as indicated by an arrow.

The drive unit 13 can include a scanner unit 31, a slide unit 32, and a bottom cover 33.

The scanner unit 31 is also referred to as a pullback unit. The scanner unit 31 is connected to the image processing device 11 via the cable 12. The scanner unit 31 includes a probe connection section 34 connected to the probe 20, and a scanner motor 35 which is a drive source for rotating the drive shaft 21.

The probe connection section 34 is freely detachably connected to the probe 20 through an insertion port 36 of the hub 22 disposed at the proximal end of the probe 20. Inside the hub 22, a proximal end of the drive shaft 21 is rotatably supported, and a rotational force of the scanner motor 35 is transmitted to the drive shaft 21. A signal is transmitted and received between the drive shaft 21 and the image processing device 11 via the cable 12. In the image processing device 11, generation of a tomographic image of a body lumen and image processing are executed based on the signal transmitted from the drive shaft 21.

The slide unit 32 is mounted with the scanner unit 31 in a manner of being capable of moving forward and backward, and is mechanically and electrically connected to the scanner unit 31. The slide unit 32 includes a probe clamp section 37, a slide motor 38, and a switch group 39.

The probe clamp section 37 is disposed coaxially with the probe connection section 34, distal of the probe connection section 34, and supports the probe 20 to be connected to the probe connection section 34.

The slide motor 38 is a drive source that generates a driving force in the axial direction. The scanner unit 31 moves forward and backward when driven by the slide motor 38, and the drive shaft 21 moves forward and backward in the axial direction accordingly. The slide motor 38 can be, for example, a servo motor.

The switch group 39 can include, for example, a forward switch and a pull-back switch that are pressed when the scanner unit 31 is to be moved forward or backward, and a scan switch that is pressed when image drawing is to be started or ended. Various switches are included in the switch group 39 as necessary without being limited to the example here.

When the forward switch is pressed, the slide motor 38 rotates forward, and the scanner unit 31 moves forward. Meanwhile, when the pull-back switch is pressed, the slide motor 38 rotates backward, and the scanner unit 31 moves backward.

When the scan switch is pressed, the image drawing is started, the scanner motor 35 is driven, and the slide motor 38 is driven to move the scanner unit 31 backward. The user such as the operator connects the probe 20 to the scanner unit 31 in advance, such that the drive shaft 21 rotates and moves toward the proximal side in the axial direction upon the start of the image drawing. When the scan switch is pressed again, the scanner motor 35 and the slide motor 38 are stopped, and the image drawing is ended.

The bottom cover 33 covers a bottom and an entire circumference of a side surface on a bottom side of the slide unit 32, and is capable of moving toward and away from the bottom of the slide unit 32.

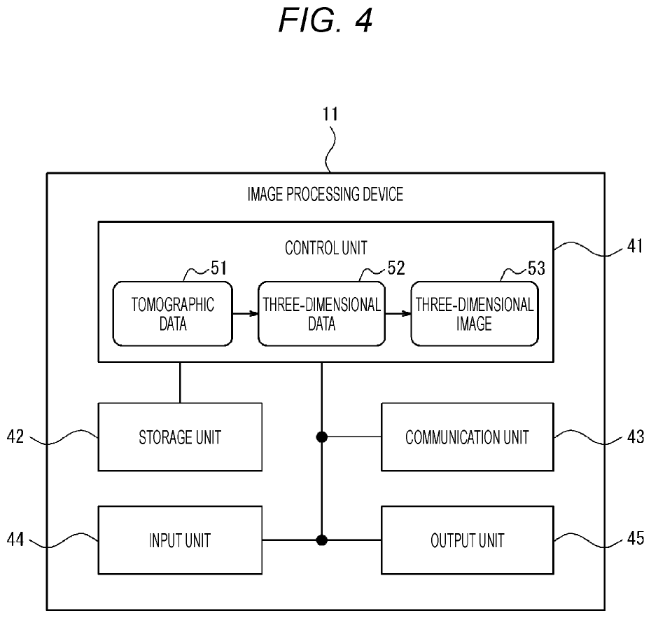

A configuration of the image processing device 11 will be described with reference to FIG. 4.

The image processing device 11 includes a control unit 41, a storage unit 42, a communication unit 43, an input unit 44, and an output unit 45.

The control unit 41 includes at least one processor, at least one programmable circuit, at least one dedicated circuit, or any combination of the at least one processor, the at least one programmable circuit, and the at least one dedicated circuit. The processor is a general-purpose processor such as a central processing unit (CPU) or graphics processing unit (GPU), or a dedicated processor specialized for specific processing. “The programmable circuit can be, for example, a field-programmable gate array (FPGA). The dedicated circuit can be, for example, an application specific integrated circuit (ASIC). The control unit 41 executes processing related to an operation of the image processing device 11 while controlling each unit of the image processing system 10 including the image processing device 11.

The storage unit 42 includes at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). “The RAM can be, for example, a static random access memory (SRAM) or a dynamic random access memory (DRAM). The ROM can be, for example, an electrically erasable programmable read only memory (EEPROM). The storage unit 42 functions as, for example, a main storage device, an auxiliary storage device, or a cache memory. The storage unit 42 stores data used for the operation of the image processing device 11, such as the tomographic data 51, and data obtained by the operation of the image processing device 11, such as the three-dimensional data 52 and the three-dimensional image 53.

The communication unit 43 includes at least one communication interface. The communication interface can be, for example, a wired local area network (LAN) interface, a wireless LAN interface, or an image diagnostic interface for receiving IVUS signals and executing analog to digital (A/D) conversion for the IVUS signals. The communication unit 43 receives data used for the operation of the image processing device 11 and transmits data obtained by the operation of the image processing device 11. In the present embodiment, the drive unit 13 is connected to the image diagnostic interface included in the communication unit 43.

The input unit 44 includes at least one input interface. The input interface can be, for example, a USB interface, a high-definition multimedia interface (HDMI®) interface, or an interface compatible with a short-range wireless communication standard such as Bluetooth®. The input unit 44 receives an operation by the user such as an operation of inputting data used for the operation of the image processing device 11. In the present embodiment, the keyboard 14 and the mouse 15 are connected to the USB interface or the interface compatible with short-range wireless communication included in the input unit 44. When a touch screen is disposed integrally with the display 16, the display 16 may be connected to the USB interface or the HDMI interface included in the input unit 44.

The output unit 45 includes at least one output interface. The output interface can be, for example, a USB interface, an HDMI interface, or an interface compatible with a short-range wireless communication standard such as Bluetooth. The output unit 45 outputs data obtained by the operation of the image processing device 11. In the present embodiment, the display 16 is connected to the USB interface or the HDMI interface included in the output unit 45.

A function of the image processing device 11 is implemented by executing an image processing program according to the present embodiment by the processor corresponding to the control unit 41. That is, the function of the image processing device 11 is implemented by software. The image processing program causes a computer to function as the image processing device 11 by causing the computer to execute the operation of the image processing device 11. That is, the computer functions as the image processing device 11 by executing the operation of the image processing device 11 according to the image processing program.

The program may be stored in a non-transitory computer-readable medium in advance. The non-transitory computer-readable medium can be, for example, a flash memory, a magnetic recording device, an optical disc, a magneto-optical recording medium, or a ROM. Distribution of the program is executed by, for example, selling, transferring, or lending a portable medium such as a secure digital (SD) card, a digital versatile disc (DVD), or a compact disc read only memory (CD-ROM) storing the program. The program may be distributed by storing the program in a storage of a server in advance and transferring the program from the server to another computer. The program may be provided as a program product.

For example, the computer temporarily stores, in the main storage device, the program stored in the portable medium or the program transferred from the server. Then, the computer reads, by the processor, the program stored in the main storage device, and executes, by the processor, processing according to the read program. The computer may read the program directly from the portable medium and execute the processing according to the program. Each time the program is transferred from the server to the computer, the computer may sequentially execute processing according to the received program. The processing may be executed by a so-called application service provider (ASP) type service in which the function is implemented only by execution instruction and result acquisition without transferring the program from the server to the computer. The program includes information provided for processing by an electronic computer and conforming to the program. For example, data that is not a direct command to the computer but has a property that defines the processing of the computer corresponds to the “information conforming to the program”.

The functions of the image processing device 11 may be partially or entirely implemented by the programmable circuit or the dedicated circuit corresponding to the control unit 41. That is, the functions of the image processing device 11 may be partially or entirely implemented by hardware.

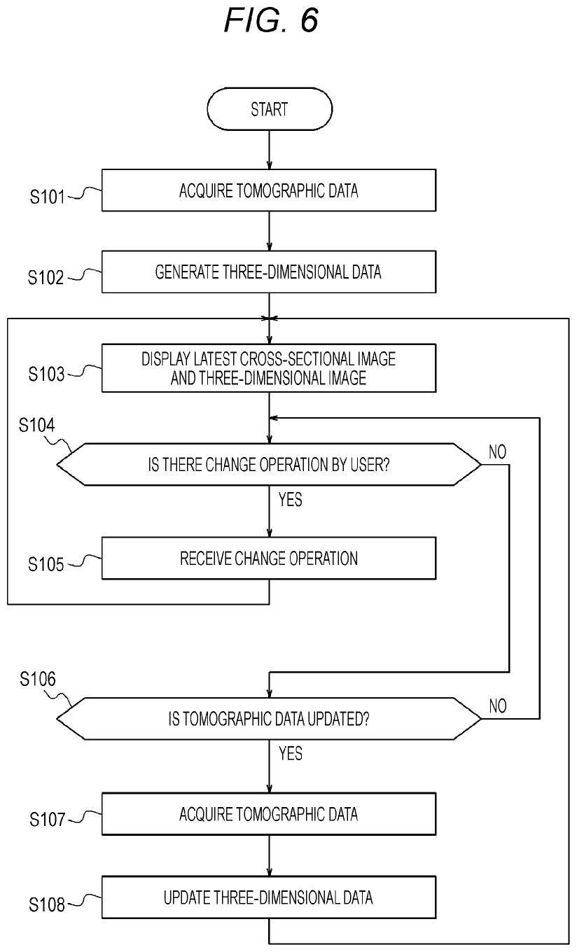

An operation of the image processing system 10 according to the present embodiment will be described with reference to FIG. 6. The operation of the image processing system 10 corresponds to an image processing method according to the present embodiment.

Before a start of a flow in FIG. 6, the probe 20 is primed by the user. Thereafter, the probe 20 is fitted into the probe connection section 34 and the probe clamp section 37 of the drive unit 13, and is connected and fixed to the drive unit 13. Then, the probe 20 is inserted into a target site in the biological tissue 60 such as the blood vessel or the heart.

In S101, the scan switch included in the switch group 39 is pressed, and a so-called pull-back operation is executed by pressing the pull-back switch included in the switch group 39. The probe 20 transmits an ultrasound wave inside the biological tissue 60 by the ultrasound transducer 25 that moves backward in the axial direction by the pull-back operation. The ultrasound transducer 25 radially transmits the ultrasound wave while moving inside the biological tissue 60. The ultrasound transducer 25 receives a reflected wave of the transmitted ultrasound wave. The probe 20 inputs a signal of the reflected wave received by the ultrasound transducer 25 to the image processing device 11. The control unit 41 of the image processing device 11 processes the input signal to sequentially generate cross-sectional images of the biological tissue 60, thereby acquiring the tomographic data 51, which includes a plurality of cross-sectional images.

Specifically, the probe 20 transmits the ultrasound wave in a plurality of directions from a rotation center to an outside by the ultrasound transducer 25 while causing the ultrasound transducer 25 to rotate in a circumferential direction and to move in the axial direction inside the biological tissue 60. The probe 20 receives the reflected wave from a reflecting object present in each of the plurality of directions inside the biological tissue 60 by the ultrasound transducer 25. The probe 20 transmits the signal of the received reflected wave to the image processing device 11 via the drive unit 13 and the cable 12. The communication unit 43 of the image processing device 11 receives the signal transmitted from the probe 20. The communication unit 43 executes A/D conversion for the received signal. The communication unit 43 inputs the A/D-converted signal to the control unit 41. The control unit 41 processes the input signal to calculate an intensity value distribution of the reflected wave from the reflecting object present in a transmission direction of the ultrasound wave of the ultrasound transducer 25. The control unit 41 sequentially generates two-dimensional images having a luminance value distribution corresponding to the calculated intensity value distribution as the cross-sectional images of the biological tissue 60, thereby acquiring the tomographic data 51 which is a data set of the cross-sectional images. The control unit 41 stores the acquired tomographic data 51 in the storage unit 42.

In the present embodiment, the signal of the reflected wave received by the ultrasound transducer 25 corresponds to raw data of the tomographic data 51, and the cross-sectional images generated by processing the signal of the reflected wave by the image processing device 11 correspond to processed data of the tomographic data 51.

In a modification of the present embodiment, the control unit 41 of the image processing device 11 may store the signal input from the probe 20 as it is (i.e., without any data conversion) in the storage unit 42 as the tomographic data 51. Alternatively, the control unit 41 may store data indicating the intensity value distribution of the reflected wave calculated by processing the signal input from the probe 20 in the storage unit 42 as the tomographic data 51. That is, the tomographic data 51 is not limited to the data set of the cross-sectional images of the biological tissue 60, and may be data representing a cross section of the biological tissue 60 at each moving position of the ultrasound transducer 25 in any format.

In a modification of the present embodiment, an ultrasound transducer that transmits the ultrasound wave in the plurality of directions without rotating may be used instead of the ultrasound transducer 25 that transmits the ultrasound wave in the plurality of directions while rotating in the circumferential direction.

In a modification of the present embodiment, the tomographic data 51 may be acquired using optical frequency domain imaging (OFDI) or optical coherence tomography (OCT) instead of being acquired using IVUS. When OFDI or OCT is used, as a sensor that acquires the tomographic data 51 while moving in the lumen 61 of the biological tissue 60, a sensor that acquires the tomographic data 51 by emitting light in the lumen 61 of the biological tissue 60 is used instead of the ultrasound transducer 25 that acquires the tomographic data 51 by transmitting the ultrasound wave in the lumen 61 of the biological tissue 60.

In a modification of the present embodiment, instead of the image processing device 11 generating the data set of the cross-sectional images of the biological tissue 60, another device may generate a similar data set, and the image processing device 11 may acquire the data set from the other device. That is, instead of the control unit 41 of the image processing device 11 processing the IVUS signal to generate the cross-sectional images of the biological tissue 60, another device may process the IVUS signal to generate the cross-sectional images of the biological tissue 60 and input the generated cross-sectional images to the image processing device 11.

In S102, the control unit 41 of the image processing device 11 generates the three-dimensional data 52 of the biological tissue 60 based on the tomographic data 51 acquired in S101. That is, the control unit 41 generates the three-dimensional data 52 based on the tomographic data 51 acquired by the sensor. Note that at this time, if already generated three-dimensional data 52 is present, it is preferable to update only data at a location corresponding to the updated tomographic data 51, instead of regenerating all pieces of the three-dimensional data 52 from the beginning. Accordingly, a data processing amount when generating the three-dimensional data 52 can be reduced, and a real-time property of the three-dimensional image 53 in the subsequent S103 can be improved.

Specifically, the control unit 41 of the image processing device 11 generates the three-dimensional data 52 of the biological tissue 60 by stacking the cross-sectional images of the biological tissue 60 included in the tomographic data 51 stored in the storage unit 42, and converting the same into three-dimensional data. As a method of three-dimensional conversion, any method among a rendering method such as surface rendering or volume rendering, and various types of processing such as texture mapping including environment mapping, and bump mapping, which are associated with the rendering method, can be used. The control unit 41 stores the generated three-dimensional data 52 in the storage unit 42.

When the catheter 63 different from an IVUS catheter, such as an ablation catheter, is inserted into the biological tissue 60, the tomographic data 51 includes data of the catheter 63, similarly to the data of the biological tissue 60. Therefore, in S102, the three-dimensional data 52 generated by the control unit 41 also includes the data of the catheter 63 similarly to the data of the biological tissue 60.

The control unit 41 of the image processing device 11 classifies a pixel group of the cross-sectional images included in the tomographic data 51 acquired in S101 into two or more classes. These two or more classes include at least a class of “tissue” to which the biological tissue 60 belongs and a class of “catheter” to which the catheter 63 belongs, and may further include a class of “blood cell”, a class of “medical device” other than “catheter” such as a guide wire, a class of “indwelling object” of an indwelling stent or the like, or a class of “lesion” of lime, plaque, or the like. As a classification method, any method may be used, but in the present embodiment, a method of classifying the pixel group of the cross-sectional images by a trained model can be used. The trained model can be trained such that a region corresponding to each class can be detected from a cross-sectional image of IVUS as a sample by performing machine learning in advance.

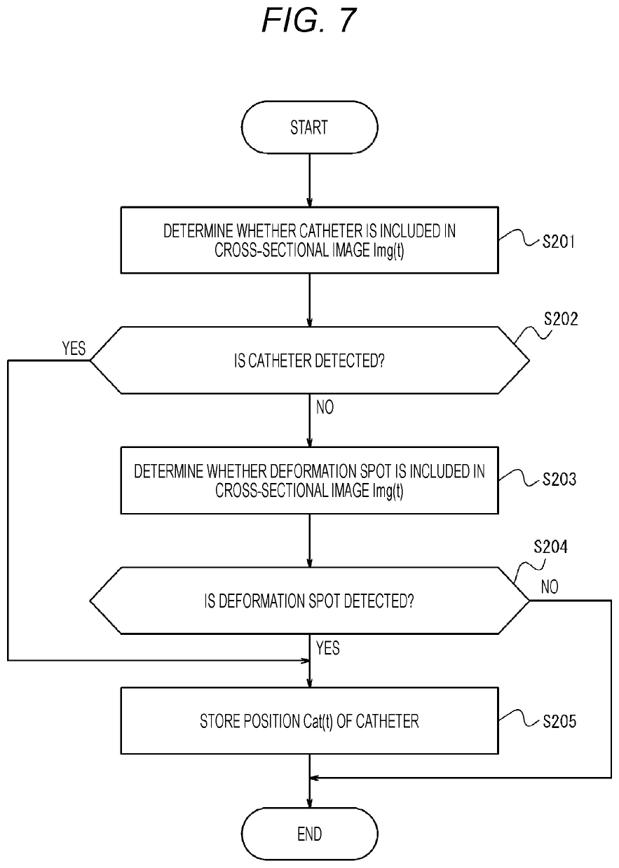

A procedure of processing further executed in S102 will be described with reference to FIG. 7. This procedure is executed each time a cross-sectional image Img(t) obtained at a time t using the sensor is input.

When receiving the input of the cross-sectional image Img(t), the control unit 41 of the image processing device 11 determines, in S201, whether the catheter 63 is included in the cross-sectional image Img(t). Specifically, the control unit 41 determines whether there is a pixel classified into the class of “catheter” in a pixel group of the cross-sectional image Img(t), thereby determining whether the catheter 63 is included in the cross-sectional image Img(t).

When it is determined that the catheter 63 is included in the cross-sectional image Img(t), that is, when the catheter 63 is detected from the cross-sectional image Img(t), the control unit 41 of the image processing device 11 specifies, in S202, a position Cat(t) of the catheter 63 in the three-dimensional space based on a position of the catheter 63 in the cross-sectional image Img(t). Specifically, the control unit 41 calculates three-dimensional coordinates corresponding to the pixel classified into the class of “catheter” as the position Cat(t) of the catheter 63. Then, in S205, the control unit 41 stores the position Cat(t) of the catheter 63 in the storage unit 42. Specifically, the control unit 41 adds data indicating the position Cat(t) of the catheter 63 to the three-dimensional data 52 stored in the storage unit 42 as data of the catheter 63.

When it is determined that the catheter 63 is not included in the cross-sectional image Img(t), that is, when the catheter 63 is not detected from the cross-sectional image Img(t), the control unit 41 of the image processing device 11 proceeds from S202 to S203. The control unit 41 determines, in S203, whether the deformation spot 69 that is on the inner surface 65 of the biological tissue 60 and that has been deformed by pressing the catheter 63 inserted into the lumen 61 is included in the cross-sectional image Img(t). Specifically, the control unit 41 selects at least one pixel Ps from the pixel group of the cross-sectional image Img(t) and determines whether the selected pixel Ps corresponds to the deformation spot 69, thereby determining whether the deformation spot 69 is included in the cross-sectional image Img(t).

When it is determined that the deformation spot 69 is included in the cross-sectional image Img(t), that is, when the deformation spot 69 is detected from the cross-sectional image Img(t), the control unit 41 of the image processing device 11 specifies, in S204, the position Cat(t) of the catheter 63 in the three-dimensional space based on a position of the deformation spot 69 in the cross-sectional image Img(t). Specifically, the control unit 41 calculates three-dimensional coordinates corresponding to the pixel determined in S203 to correspond to the deformation spot 69 as the position Cat(t) of the catheter 63. Then, in S205, the control unit 41 stores the position Cat(t) of the catheter 63 in the storage unit 42. Specifically, the control unit 41 adds data indicating the position Cat(t) of the catheter 63 to the three-dimensional data 52 stored in the storage unit 42 as data of the catheter 63.

When it is determined that the deformation spot 69 is not included in the cross-sectional image Img(t), that is, when the deformation spot 69 is not detected from the cross-sectional image Img(t), the control unit 41 of the image processing device 11 ends the procedure of FIG. 7.

As a method of selecting the pixel Ps in S203, for example, any one of the following first to third methods can be used.

The first method is a method of selecting a corner on a rule basis. The first method includes the following:

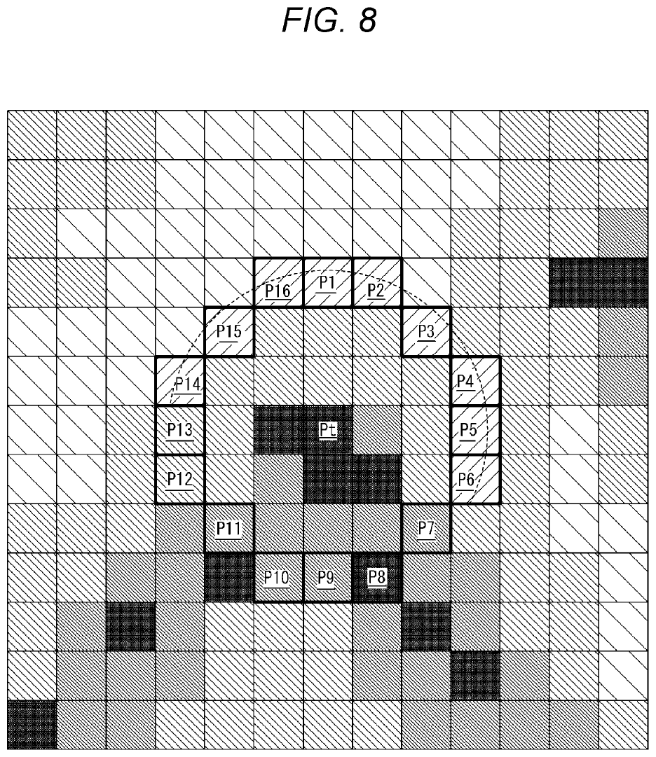

1. For each pixel of the cross-sectional image Img(t), a pixel group present on a circumference at a constant distance from a target pixel Pt is extracted. FIG. 8 illustrates an example in which the distance is three pixels.

2. It is determined whether each pixel is a pixel similar to the target pixel Pt. For example, it is determined whether each pixel is as black or white as the target pixel Pt.

3. It is determined whether the pixels regarded to be similar continuously occupy, for example, a range of 45 degrees to 135 degrees of the circumference as a result of the determination. That is, a fan-shaped region of a range of 45 degrees or more and 135 degrees or less is detected. A lower limit value of the angular range is not limited to 45 degrees, and may be any value greater than 0 degrees. An upper limit value of the angular range is not limited to 135 degrees, and may be any value smaller than 180 degrees.

4. When the fan-shaped region matching the condition is detected, the target pixel Pt is regarded as a corner. That is, the target pixel Pt is selected as the pixel Ps. A pixel adjacent to the target pixel Pt may be further selected as the pixel Ps.

A known corner detection algorithm may be applied. For an example of such an algorithm, refer to the following website:
https://docs.opencv.org/master/df/d0c/tutorial_py_fast.html

When the first method is adopted, for each pixel of the cross-sectional image Img(t), the control unit 41 of the image processing device 11 calculates similarity with the target pixel Pt for each pixel present on the circumference at a constant distance from the target pixel Pt. The similarity is calculated by comparing characteristics of each pixel such as luminance of each pixel or a class into which each pixel is classified. The control unit 41 selects the target pixel Pt as the pixel Ps when an angle at which the pixels having the calculated similarity equal to or greater than a reference value continuously present on the circumference is within a reference range. The reference value can be set to any value in advance, and can be adjusted as appropriate. The reference range may be any range of greater than 0 degrees and smaller than 180 degrees, but can be set in advance to, for example, the range of 45 degrees or more and 135 degrees or less.

In the example of FIG. 8, it is assumed that, among pixels P1, . . . , P16 present on the circumference where a distance from the target pixel Pt is three pixels, five pixels P7, . . . , P11 are similar to the target pixel Pt by a reference value or more. It is assumed that these five pixels P7, . . . , P11 are continuously present on the circumference, and an angle of an arc formed by the pixels P7, . . . , P11 is in the range of 45 degrees or more and 135 degrees or less. In this case, the target pixel Pt is selected as the pixel Ps.

The second method is also a method of selecting a corner on a rule basis. The second method includes the following:

1. A boundary line of a region corresponding to the lumen 61 of the cross-sectional image Img(t) is extracted.

2. For each pixel on the boundary line, the target pixel Pt is set as a starting point.

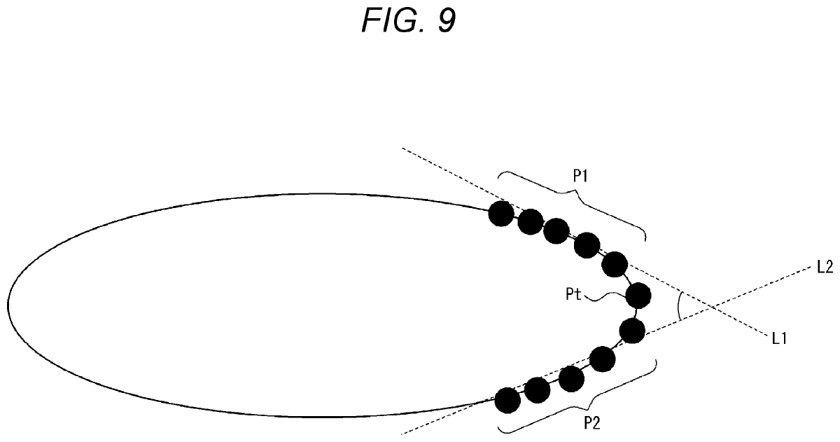

3. A certain number of nearest points present on the boundary line in a counterclockwise direction from the starting point is extracted as a point group P1. FIG. 9 illustrates an example of extracting five points.

4. A certain number of nearest points present on the boundary line in a clockwise direction from the starting point are extracted as a point group P2. FIG. 9 illustrates an example of extracting five points.

5. A regression line L1 and a regression line L2 are drawn for the point group P1 and the point group P2, respectively.

6. It is determined whether the smaller angle of angles formed by the regression line L1 and the regression line L2 is in a range of 30 degrees or more and 75 degrees or less. That is, a substantially V-shaped boundary line in the range of 30 degrees or more and 75 degrees or less is detected. A lower limit value of the angular range is not limited to 30 degrees, and may be any value greater than 0 degrees. An upper limit value of the angular range is not limited to 75 degrees, and may be any value smaller than 90 degrees.

7. When the substantially V-shaped boundary line matching the condition is detected, the target pixel Pt is regarded as a corner. That is, the target pixel Pt is selected as the pixel Ps. A pixel included in the point group P1 and the point group P2 may be further selected as the pixel Ps.

When the second method is adopted, for each pixel on a boundary line corresponding to the inner surface 65 of the biological tissue 60 in the pixel group of the cross-sectional image Img(t), the control unit 41 of the image processing device 11 extracts a first pixel group present on one side of the target pixel Pt on the boundary line and a second pixel group present on the other side of the target pixel Pt on the boundary line. The first pixel group corresponds to the point group P1. The second pixel group corresponds to the point group P2. When magnitude of an angle formed by a regression line L1 corresponding to the extracted first pixel group and a regression line L2 corresponding to the extracted second pixel group is within a reference range, the control unit 41 selects the target pixel Pt as the pixel Ps. The reference range may be any range of greater than 0 degrees and smaller than 90 degrees, but can be set in advance to, for example, the range of 30 degrees or more and 75 degrees or less.

In the example of FIG. 9, it is assumed that the magnitude of the angle formed by the regression line L1 corresponding to the first pixel group present on one side of the target pixel Pt on the boundary line and the regression line L2 corresponding to the second pixel group present on the other side of the target pixel Pt on the boundary line is in the range of 30 degrees or more and 75 degrees or less. In this case, the target pixel Pt is selected as the pixel Ps.

The third method is a method of selecting a corner by a neural network. The third method includes the following:

1. A machine learning algorithm Am for determining whether there is a tenting spot using at least an ultrasound image as an input is created in advance.

2. It is determined whether there is a tenting spot by the algorithm Am, using a target ultrasound image as an input.

3. When it is determined that there is a tenting spot, a region emphasized by the algorithm Am is extracted using gradient-weighted class activation mapping (Grad-CAM), for example.

4. A pixel at the center of the region emphasized by the algorithm Am is regarded as a corner. That is, the pixel at the center is selected as the pixel Ps. A pixel in a certain region including the center may be further selected as the pixel Ps.

For Grad-CAM, refer to the following website:
https://qiita.com/kinziro/items/69f996065b4a658c42e8

When the third method is adopted, the control unit 41 of the image processing device 11 inputs the cross-sectional image Img(t) to a trained model that infers, using an image obtained using the sensor as an input, a pixel corresponding to the deformation spot 69 of the input image. The trained model can be created in advance by, for example, deep learning. The control unit 41 selects the inferred pixel as the pixel Ps.

As a method of determining whether the pixel Ps corresponds to the deformation spot 69 in S203, for example, the following first method or second method can be used.

The first method is a method of determining whether a corner is a tenting spot in an orthogonal coordinate system based on past catheter information. The first method includes the following:

1. Information indicating a position Pc of the catheter 63 in a cross-sectional image Img(u) obtained at a time u when the catheter 63 is detected last is referred to. The time u can be, for example, a time t−1. When a distance in the Z direction between a cross section represented by the cross-sectional image Img(u) and the cross section 64 represented by the cross-sectional image Img(t) is a certain distance or more, information indicating a position of the catheter 63 detected before the time u may be referred to instead.

2. A line Lt connecting a point Pa regarded as a corner and the centroid Pb of the cross section 64 or the center of the cross-sectional image Img(t) is drawn.

3. A line Lc connecting the position Pc and the centroid Pb of the cross section 64 or the center of the cross-sectional image Img(t) is drawn.

4. It is determined whether the smaller angle of angles formed by the line Lt and the line Lc is 30 degrees or less. When the angle is 30 degrees or less, it is determined that the point Pa is a tenting spot. The threshold of the angle is not limited to 30 degrees, and may be any value smaller than 45 degrees.

When the first method is adopted, the control unit 41 of the image processing device 11 determines whether the selected pixel Ps corresponds to the deformation spot 69 according to a positional relationship between the selected pixel Ps and the catheter 63 in the cross-sectional image Img(u) including the catheter 63 different from the cross-sectional image Img(t). In the example of FIG. 2, the position indicated by the mark Mc corresponds to the position Pc of the catheter 63 in the cross-sectional image Img(u). The position indicated by the mark Mt corresponds to the position of the pixel Ps when it is determined that the pixel Ps corresponds to the tenting spot, that is, the point Pa. This means that magnitude of an angle formed by a line Lt connecting the mark Mt and the centroid Pb and a line Lc connecting the mark Mc and the centroid Pb is 30 degrees or less.

The first method may be a method of determining whether a corner is a tenting spot in an orthogonal coordinate system based on catheter information at a time after the time t. For example, when the catheter 63 is not detected at the time t and a corner that is a candidate for a tenting spot is obtained, and the catheter 63 is detected at the time t+1, whether the corner is the tenting spot may be determined by a similar method retroactively at the time t.

The first method may be a method of determining whether a corner is a tenting spot in a polar coordinate system based on past catheter information. In that case, the first method includes the following:

1. The information indicating the position Pc of the catheter 63 in the cross-sectional image Img(u) obtained at the time u when the catheter 63 is detected last is referred to.

2. The position Pc is compared with the point Pa regarded as a corner, and it is determined whether an absolute value of a difference between angle components is 30 degrees or less. When the absolute value of the difference between the angle components is 30 degrees or less, it is determined that the point Pa is a tenting spot. The threshold of the angle is not limited to 30 degrees, and may be any value smaller than 45 degrees.

The second method is a method of determining whether a corner is a tenting spot based on continuity of tenting spots. The second method includes the following:

1. It is determined whether there are a point regarded as a corner in a cross-sectional image Img(t−1) and a point regarded as a corner in a cross-sectional image Img(t−2) in 3 mm around the point Pa regarded as a corner. The peripheral distance is not limited to 3 mm, and may be, for example, about 4 mm or about 5 mm. The peripheral distance may be replaced by a radius distance or an angular difference. Instead of determining whether there are corners continuously in three frames, it may be determined whether there are corners continuously in two frames, or whether there are corners continuously in four or more frames.

2. It is determined that the point Pa is a tenting spot when there are both the point regarded as a corner in the cross-sectional image Img(t−1) and the point regarded as a corner in the cross-sectional image Img(t−2) in 3 mm around the point Pa.

When the second method is adopted, the control unit 41 of the image processing device 11 selects at least one pixel Ps from a pixel group of each of two or more cross-sectional images continuously obtained using the sensor. The control unit 41 determines whether the selected pixel Ps corresponds to the deformation spot 69 according to a difference in position of the selected pixel Ps between the two or more cross-sectional images.

The second method may be combined with the first method. In that case, the second method includes the following:

1. It is determined whether there are a point regarded as a tenting spot in the cross-sectional image Img(t−1) and a point regarded as a tenting spot in the cross-sectional image Img(t−2) in 3 mm around the point Pa regarded as a tenting spot in the first method. The peripheral distance is not limited to 3 mm, and may be, for example, about 4 mm or about 5 mm. The peripheral distance may be replaced by a radius distance or an angular difference. Instead of determining whether there are tenting spots continuously in three frames, it may be determined whether there are tenting spots continuously in two frames, or whether there are tenting spots continuously in four or more frames.

2. It is determined that the point Pa is a tenting spot when there are both the point regarded as a tenting spot in the cross-sectional image Img(t−1) and the point regarded as a tenting spot in the cross-sectional image Img(t−2) in 3 mm around the point Pa.

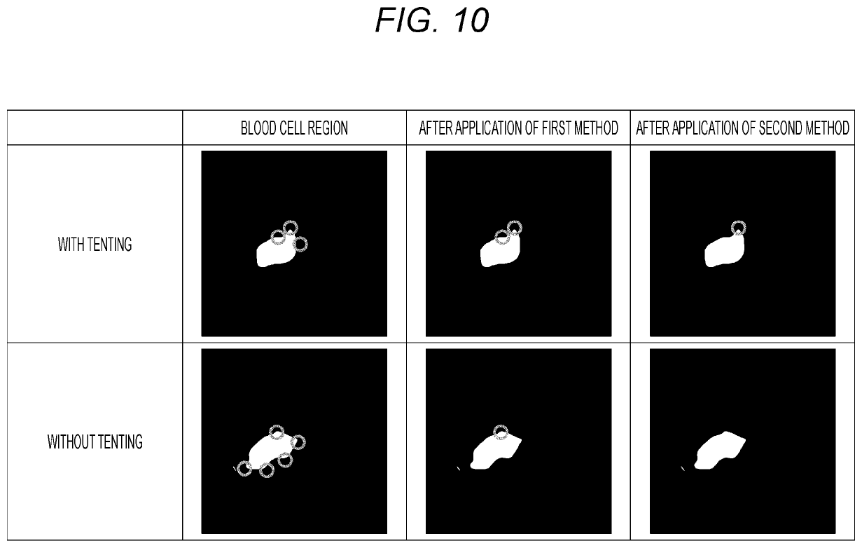

By adopting, as the first method, the method of determining whether a corner is a tenting spot in the orthogonal coordinate system based on past catheter information, and combining the second method with the first method, accuracy of tenting spot detection can be greatly improved as illustrated in FIG. 10. In the example of FIG. 10, a peripheral edge portion of a pixel group classified into the class of “blood cell” is set as a boundary line, and several corners are detected from the boundary line. By applying the first method, some corners are excluded from the several corners. Then, by further applying the second method, when there is a tenting spot, a position of the tenting spot can be specified, and when there is no tenting spot, it can be determined that there is no tenting spot.

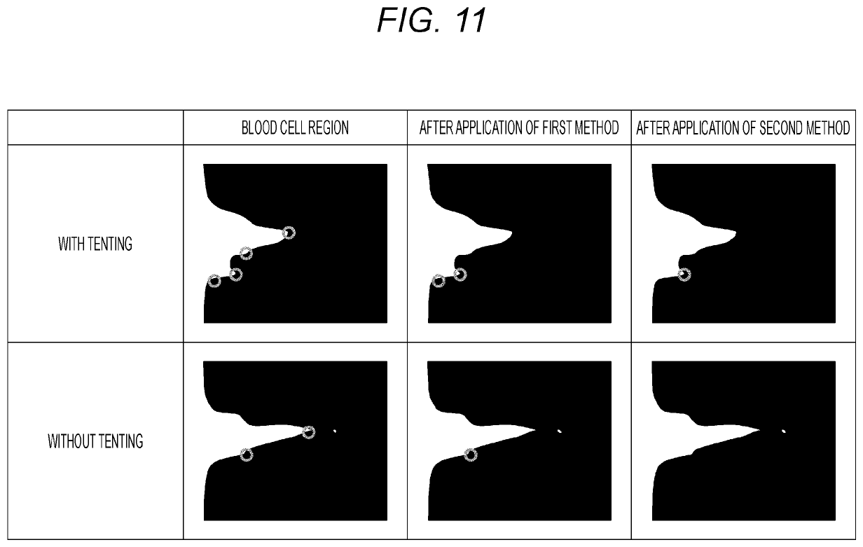

Also by adopting, as the first method, the method of determining whether a corner is a tenting spot in the polar coordinate system based on past catheter information, and combining the second method with the first method, the accuracy of the tenting spot detection can be greatly improved as illustrated in FIG. 11. In the example of FIG. 11, a peripheral edge portion of a pixel group classified into the class of “blood cell” is set as a boundary line, and several corners are detected from the boundary line. By applying the first method, one or two corners are excluded from the several corners. Then, by further applying the second method, when there is a tenting spot, a position of the tenting spot is specified, and when there is no tenting spot, it can be determined that there is no tenting spot.

In a modification of the present embodiment, when a corner is detected at the same position or a close position continuously for a certain distance, such as 10 mm in the Z direction, the control unit 41 of the image processing device 11 may determine that the corner is not a tenting spot. Alternatively, when a corner is detected at the same position or a close position continuously for a certain period of time, such as several seconds, the control unit 41 may determine that the corner is not a tenting spot.

In a modification of the present embodiment, in S203, the control unit 41 of the image processing device 11 may determine whether the deformation spot 69 is included in the cross-sectional image Img(t) without selecting the pixel Ps. In such a modification, the control unit 41 inputs the cross-sectional image Img(t) to a trained model that infers, using an image obtained using the sensor as an input, whether the deformation spot 69 is included in the input image. The control unit 41 determines whether the deformation spot 69 is included in the cross-sectional image Img(t) by referring to an obtained inference result. For example, the same machine learning algorithm Am as that of the third method may be created in advance. In that case, it is determined whether there is a tenting spot by the algorithm Am, using a target ultrasound image as an input. When it is determined that there is a tenting spot, a region emphasized by the algorithm Am is extracted using Grad-CAM. A pixel at the center of the region emphasized by the algorithm Am is regarded as a tenting spot.

In a further modification of this modification, the control unit 41 of the image processing device 11 may input the cross-sectional image Img(t) to a trained model that infers, using an image obtained using the sensor as an input, data indicating a position of the deformation spot 69 in the input image. In such a modification, the control unit 41 determines whether the deformation spot 69 is included in the cross-sectional image Img(t) by referring to an obtained inference result. For example, when the machine learning algorithm Am is created, data indicating a position of a tenting spot may be included in teacher data.

In S103, the control unit 41 of the image processing device 11 causes the display 16 to display the three-dimensional data 52 generated in S102 as the three-dimensional image 53. At this time point, the control unit 41 may set an angle for displaying the three-dimensional image 53 to any angle. The control unit 41 causes the display 16 to display the latest cross-sectional image 54 included in the tomographic data 51 acquired in S101 together with the three-dimensional image 53.

Specifically, the control unit 41 of the image processing device 11 generates the three-dimensional image 53 based on the three-dimensional data 52 stored in the storage unit 42. The three-dimensional image 53 includes a three-dimensional object group including an object representing the biological tissue 60 in the three-dimensional space, an object representing the catheter 63 in the three-dimensional space, and the like. That is, the control unit 41 generates a three-dimensional object of the biological tissue 60 from the data of the biological tissue 60 stored in the storage unit 42, and generates a three-dimensional object of the catheter 63 from the data of the catheter 63 stored in the storage unit 42. The control unit 41 causes the display 16 to display the latest cross-sectional image 54 among the cross-sectional images of the biological tissue 60 included in the tomographic data 51 stored in the storage unit 42 and the generated three-dimensional image 53 via the output unit 45.

In S104, if there is an operation of setting the angle for displaying the three-dimensional image 53 as a change operation by the user, processing of S105 is executed. If there is no change operation by the user, processing of S106 is executed.

In S105, the control unit 41 of the image processing device 11 receives, via the input unit 44, the operation of setting the angle for displaying the three-dimensional image 53. The control unit 41 adjusts the angle for displaying the three-dimensional image 53 to the set angle. In S103, the control unit 41 causes the display 16 to display the three-dimensional image 53 at the angle set in S105.

Specifically, the control unit 41 of the image processing device 11 receives, via the input unit 44, an operation by the user of rotating the three-dimensional image 53 displayed on the display 16 by using the keyboard 14, the mouse 15, or the touch screen disposed integrally with the display 16. The control unit 41 interactively adjusts the angle for displaying the three-dimensional image 53 on the display 16 according to the operation by the user. Alternatively, the control unit 41 receives, via the input unit 44, an operation by the user of inputting a numerical value of the angle for displaying the three-dimensional image 53 by using the keyboard 14, the mouse 15, or the touch screen disposed integrally with the display 16. The control unit 41 adjusts the angle for displaying the three-dimensional image 53 on the display 16 in accordance with the input numerical value.

In S106, if the tomographic data 51 is updated, processing of S107 and S108 is executed. If the tomographic data 51 is not updated, the presence or absence of the change operation by the user is confirmed again in S104.

In S107, similarly to the processing of S101, the control unit 41 of the image processing device 11 processes the signal input from the probe 20 to newly generate a cross-sectional image 54 of the biological tissue 60, thereby acquiring the tomographic data 51 including at least one new cross-sectional image 54.

In S108, the control unit 41 of the image processing device 11 updates the three-dimensional data 52 of the biological tissue 60 based on the tomographic data 51 acquired in S107. That is, the control unit 41 updates the three-dimensional data 52 based on the tomographic data 51 acquired by the sensor. In S108, the operation of FIG. 7 is also performed. The procedure illustrated in FIG. 7 is executed each time the cross-sectional image Img(t) obtained at the time t using the sensor is input. Then, in S103, the control unit 41 causes the display 16 to display the three-dimensional data 52 updated in S108 as the three-dimensional image 53. The control unit 41 causes the display 16 to display the latest cross-sectional image 54 included in the tomographic data 51 acquired in S107 together with the three-dimensional image 53. In S108, it is preferable to update only data at a location corresponding to the updated tomographic data 51. Accordingly, the data processing amount when generating the three-dimensional data 52 can be reduced, and the real-time property of the three-dimensional image 53 can be improved in S108.

As described above, in the present embodiment, when receiving an input of at least one cross-sectional image obtained using the sensor that moves in the lumen 61 of the biological tissue 60, the control unit 41 of the image processing device 11 determines whether the deformation spot 69 that is on the inner surface 65 of the biological tissue 60 and that has been deformed by pressing the catheter 63 inserted into the lumen 61 is included in the at least one cross-sectional image. When it is determined that the deformation spot 69 is included in the at least one cross-sectional image, the control unit 41 specifies a position of the catheter 63 based on a position of the deformation spot 69. Therefore, according to the present embodiment, it is possible to specify the position the catheter 63 even when it is difficult to detect the catheter 63 from the image obtained using the sensor. For example, it is possible to construct an artificial intelligence (AI) that detects a recess of a tenting spot and specifies the tenting spot.

The present disclosure is not limited to the above-described embodiment. For example, two or more blocks described in the block diagram may be integrated, or one block may be divided. Instead of executing two or more steps described in the flowchart in time series according to the description, the steps may be executed in parallel or in a different order according to the processing capability of the device that executes each step or as necessary. In addition, modifications can be made without departing from a gist of the present disclosure.

The detailed description above describes embodiments of an image processing device, an image processing system, an image processing method, and an image processing program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing device comprising:
a control unit configured to:
determine, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image; and
specify a position of the medical device based on a position of the deformation spot when it is determined that the deformation spot is included in the at least one cross-sectional image.

2. The image processing device according to claim 1, wherein the control unit is configured to:
determine, when the input of the at least one cross-sectional image is received, whether the medical device is included in the at least one cross-sectional image; and
determine, when it is determined that the medical device is not included in the at least one cross-sectional image, whether the deformation spot is included in the at least one cross-sectional image.

3. The image processing device according to claim 2, wherein,
when it is determined that the medical device is not included in the at least one cross-sectional image and it is determined that the deformation spot is included in the at least one cross-sectional image, the control unit is configured to specify a position of the medical device in a three-dimensional space based on a position of the deformation spot in the at least one cross-sectional image; and
when it is determined that the medical device is included in the at least one cross-sectional image, the control unit is configured to specify the position of the medical device in the three-dimensional space based on the position of the medical device in the at least one cross-sectional image.

4. The image processing device according to claim 3, wherein the control unit is configured to:
cause a display to display a three-dimensional object group including an object representing the biological tissue in the three-dimensional space and an object representing the medical device in the three-dimensional space.

5. The image processing device according to claim 1, wherein the control unit is configured to:
determine whether the deformation spot is included in the at least one cross-sectional image by selecting at least one pixel from a pixel group of the at least one cross-sectional image; and
determine whether the selected pixel corresponds to the deformation spot.

6. The image processing device according to claim 5, wherein the control unit is configured to:
calculate, for each pixel of the at least one cross-sectional image, similarity with a target pixel for each pixel present on a circumference at a constant distance from the target pixel; and
select the target pixel as the at least one pixel when an angle at which pixels having the calculated similarity equal to or greater than a reference value continuously present on the circumference is within a reference range.

7. The image processing device according to claim 5, wherein the control unit is configured to:
extract, for each pixel on a boundary line corresponding to the inner surface of the biological tissue in the pixel group of the at least one cross-sectional image, a first pixel group present on one side of a target pixel on a boundary line and a second pixel group present on an other side of the target pixel on the boundary line; and
select the target pixel as the at least one pixel when magnitude of an angle formed by a regression line corresponding to the extracted first pixel group and a regression line corresponding to the extracted second pixel group is within a reference range.

8. The image processing device according to claim 5, wherein the control unit is configured to:
input the at least one cross-sectional image to a trained model that infers, using an image obtained using the sensor as an input, a pixel corresponding to the deformation spot of the input image; and
select the inferred pixel as the at least one pixel.

9. The image processing device according to claim 5, wherein the control unit is configured to:
determine whether the selected pixel corresponds to the deformation spot according to a positional relationship between the selected pixel and the medical device in a cross-sectional image including the medical device different from the at least one cross-sectional image.

10. The image processing device according to claim 5, wherein the control unit is configured to:
select the at least one pixel from a pixel group of each of two or more cross-sectional images continuously obtained using the sensor; and
determine whether the selected pixel corresponds to the deformation spot according to a difference in position of the selected pixel between the two or more cross-sectional images.

11. The image processing device according to claim 1, wherein the control unit is configured to:
input the at least one cross-sectional image to a trained model that infers, using an image obtained using the sensor as an input, whether the deformation spot is included in the input image; and
determine whether the deformation spot is included in the at least one cross-sectional image by referring to an obtained inference result.

12. The image processing device according to claim 1, wherein the control unit is configured to:
input the at least one cross-sectional image to a trained model that infers, using an image obtained using the sensor as an input, data indicating a position of the deformation spot in the input image; and
determine whether the deformation spot is included in the at least one cross-sectional image by referring to an obtained inference result.

13. An image processing system comprising:
the image processing device according to claim 1; and
the sensor.

14. An image processing system comprising:
the image processing device according to claim 4; and
the display.

15. An image processing method comprising:
determining, by a control unit, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image; and
specifying, by the control unit, a position of the medical device based on a position of the deformation spot when it is determined that the deformation spot is included in the at least one cross-sectional image.

16. The image processing method according to claim 15, further comprising:
determining, by the control unit, when the input of the at least one cross-sectional image is received, whether the medical device is included in the at least one cross-sectional image; and
determining, by the control unit, when it is determined that the medical device is not included in the at least one cross-sectional image, whether the deformation spot is included in the at least one cross-sectional image.

17. The image processing method according to claim 16, wherein,
when it is determined that the medical device is not included in the at least one cross-sectional image and it is determined that the deformation spot is included in the at least one cross-sectional image, specifying, by the control unit, a position of the medical device in a three-dimensional space based on a position of the deformation spot in the at least one cross-sectional image;
when it is determined that the medical device is included in the at least one cross-sectional image, specifying, by the control unit, the position of the medical device in the three-dimensional space based on the position of the medical device in the at least one cross-sectional image; and
causing, by the control unit, a display to display a three-dimensional object group including an object representing the biological tissue in the three-dimensional space and an object representing the medical device in the three-dimensional space.

18. The image processing method according to claim 15, further comprising:
determining, by the control unit, whether the deformation spot is included in the at least one cross-sectional image by selecting at least one pixel from a pixel group of the at least one cross-sectional image and determining whether the selected pixel corresponds to the deformation spot;
calculating, by the control unit, for each pixel of the at least one cross-sectional image, similarity with a target pixel for each pixel present on a circumference at a constant distance from the target pixel; and
selecting, by the control unit, the target pixel as the at least one pixel when an angle at which pixels having the calculated similarity equal to or greater than a reference value continuously present on the circumference is within a reference range.

19. The image processing method according to claim 15, further comprising:
determining, by the control unit, whether the deformation spot is included in the at least one cross-sectional image by selecting at least one pixel from a pixel group of the at least one cross-sectional image and determining whether the selected pixel corresponds to the deformation spot;
extracting, by the control unit, for each pixel on a boundary line corresponding to the inner surface of the biological tissue in the pixel group of the at least one cross-sectional image, a first pixel group present on one side of a target pixel on a boundary line and a second pixel group present on the other side of the target pixel on the boundary line; and
selecting, by the control unit, the target pixel as the at least one pixel when magnitude of an angle formed by a regression line corresponding to the extracted first pixel group and a regression line corresponding to the extracted second pixel group is within a reference range.

20. A non-transitory computer-readable medium storing an image processing program for causing a computer to execute a process comprising:
determining, when an input is received of at least one cross-sectional image obtained using a sensor configured to move in a lumen of a biological tissue, whether a deformation spot that is on an inner surface of the biological tissue and that has been deformed by pressing an elongated medical device inserted into the lumen is included in the at least one cross-sectional image; and
specifying a position of the medical device based on a position of the deformation spot when it is determined that the deformation spot is included in the at least one cross-sectional image.
